SEQUENCE LISTING (4 sequences)

SEQ ID NO: 1 - DNA, 30 bp, synthetic construct
  source 1..30: mol_type = other DNA
cttgaccttgccacagaggactattagagg

SEQ ID NO: 2 - DNA, 30 bp, synthetic construct
  source 1..30: mol_type = other DNA
cagtgacacttaccgcattgacaagcacgc

SEQ ID NO: 3 - DNA, 30 bp, synthetic construct
  source 1..30: mol_type = other DNA
cctcgatatacagaccgataaaacacatgc

SEQ ID NO: 4 - DNA, 30 bp, synthetic construct
  source 1..30: mol_type = other DNA
agtcagtcagaaacaactttggcacatatc

(19) United States
(12) Patent Application Publication
(10) Pub. No.: US 2024/0400668 A1
(43) Pub. Date: Dec. 5, 2024

(54) NON-HUMAN MAMMALIAN MODEL EXPRESSING IL-8 AND USE THEREOF

(71) Applicant: JINFENG LABORATORY, Chongqing (CN)

(72) Inventors: Haofei LIU, Chongqing (CN); Xindong LIU, Chongqing (CN)

(73) Assignee: JINFENG LABORATORY, Chongqing (CN)

(21) Appl. No.: 18/694,608

(22) PCT Filed: Jul. 29, 2022
(86) PCT No.: PCT/CN2022/109093

§ 371 Date: Mar. 22, 2024

(51) Int. Cl. C07K 16/24; A61K 39/00; A61P 35/00

(52) U.S. Cl. CPC C07K 16/244; A61P 35/00; A61K 2039/505; C07K 2317/21; C07K 2317/24; C07K 2317/565; C07K 2317/76

(57) ABSTRACT

Provided is a non-human mammalian model expressing IL-8 and use thereof. Specifically. provided is a non-human mammalian model expressing IL-8. The non-human mammalian model expressing IL-8 can be better used to prepare a tumor-bearing non-human mammalian model expressing IL-8 which can be used for studying the effect of IL-8 on tumors and screening or identifying potential therapeutic agents for the prevention and/or treatment of tumors.

### INCORPORATION BY REFERENCE

The sequence listing in TXT format is incorporated herein by reference in its entirety. Name of File: NON-HUMAN MAMMALIAN MODEL EXPRESSING IL-8 AND USE THEREOF
Date of Creation: 14 Dec. 2023
Size of File: 4.18KB

### TECHNICAL FIELD

The present invention relates to the field of biotechnology, and in particular to non-human mammalian model expressing il-8 and use thereof.

### BACKGROUND TECHNIQUE

Tumor is a disease that seriously threatens human health, and the research and development of anti-tumor drugs has always been a hot spot.

Tumor-bearing animal models can be used for the development of anti-tumor drugs, and can reduce the cost of anti-tumor drug development. However, existing tumor-bearing animal models still have many disadvantages, such as the slow growth of tumor and tumor blood vessel, thereby increasing the preparing cost of tumor-bearing animal models; the tumor-bearing animal models are difficult to be effectively for anticancer drug screening, etc. thus limiting the research and development of tumor drugs.

Therefore, there is a need in the art to prepare an animal model for promoting the development of anti-tumor drugs.

### SUMMARY OF THE INVENTION

The object of the present invention is to provide a non-human mammalian model expressing IL-8, the non-human mammalian model expressing IL-8 can be better used to prepare tumor animal model, and the tumor animal model can be used for studying the effect of IL-8 on tumor and to screen or identify potential therapeutic agents to prevent and/or treat tumors.

Another object of the present invention is to provide a use of an IL-8 inhibitor for enhancing the anti-tumor effect of a PD-1 inhibitor.

In the first aspect of the invention, a non-human mammalian model expressing IL-8 is provided.

In another preferred embodiment, the IL-8 comprises human IL-8 or primate IL-8.

In another preferred embodiment, the non-human mammalian is rodent.

In another preferred embodiment, the non-human mammalian comprises mice, rat, rabbit and/or monkey.

In another preferred embodiment, the mice comprises C57BL/6 mice.

In another preferred embodiment, the IL-8 comprises serum IL-8 or tissue IL-8.

In another preferred embodiment, the non-human mammalian model expressing IL-8 is heterozygous or homozygous.

In the second aspect of the invention, a method for preparing the non-human mammalian model expressing IL-8 according to the first aspect of the present invention is provided, the method comprises:
subjecting the non-human mammalian to express IL-8, thereby obtaining the non-human mammalian model expressing IL-8.

In another preferred embodiment, the method for preparing the non-human mammalian model expressing IL-8 comprises:
subjecting the non-human mammalian to having a gene expressing IL-8, thereby obtaining the non-human mammalian model expressing IL-8.

In another preferred embodiment, the method for preparing the non-human mammalian model expressing IL-8 comprises:
inserting a gene expressing IL-8 into the non-human mammalian to make the non-human mammalian express IL-8, thereby obtaining the non-human mammalian model expressing IL-8.

In another preferred embodiment, the gene expressing IL-8 is located in the genome of the non-human mammalian.

In another preferred embodiment, the gene expressing IL-8 is located at positions 3023490-3024411 of chromosome 9 of the non-human mammalian cells.

In another preferred embodiment, the method for preparing the non-human mammalian model expressing IL-8 comprises:
(a) providing the non-human mammalian cell, subjecting the cell to express IL-8, thereby obtaining the non-human mammalian cell expressing IL-8;
(b) using the non-human mammalian cell expressing IL-8 obtained in step (a) to prepare the non-human mammalian model expressing IL-8.

In another preferred embodiment, in step (a), the genome of the cell comprises a gene expressing IL-8.

In another preferred embodiment, the step (a) comprises:
providing the non-human mammalian cell, a gene expressing IL-8 was inserted into the cell, thereby obtaining the non-human mammalian cell expressing IL-8;

In another preferred embodiment, the step (a) comprises:
inserting the gene expressing IL-8 into the genome of the non-human mammalian cell by using gene insertion technology to obtain the non-human mammalian cell expressing IL-8.

In another preferred embodiment, the step (b) comprises:
(b1) subjecting the non-human mammalian cell expressing IL-8 obtained in step (a) to prepare a chimeric non-human mammalian;
(b2) mating the chimeric non-human mammal obtained in step (b1) with a normal wild-type non-human mammalian, and screening progeny to obtain a heterozygous non-human mammalians expressing IL-8;
(b3) mating the heterozygous non-human mammalians obtained in step (b2) with each other to obtain the non-human mammal model expressing IL-8.

In the third aspect of the present invention, a use of the non-human mammalian model expressing IL-8 according to the first aspect of the present invention for preparing a tumor-bearing non-human mammalian model expressing IL-8 is provided.

In another preferred embodiment, the tumor-bearing non-human mammalian model expressing IL-8 bears tumor and expresses IL-8.

In another preferred embodiment, the tumor-bearing non-human mammalian model expressing IL-8 is the non-human mammalian model expressing IL-8 which bears tumor.

In another preferred embodiment, the non-human mammalian model expressing IL-8 is according to the first aspect of the present invention.

In another preferred embodiment, the tumor comprises human tumor or non-human mammalian tumor.

In another preferred embodiment, the non-human mammalian is rodent.

In another preferred embodiment, the non-human mammalian comprises mice, rat, rabbit and/or monkey.

In another preferred embodiment, the mice include C57BL/6 mice.

In another preferred embodiment, the tumor comprises one or more of colon cancer, lung cancer, breast cancer, pancreatic cancer, brain tumor and leukemia.

In another preferred embodiment, the leukemia comprises acute myeloid leukemia M5.

In another preferred embodiment, the cell of the tumor comprises one or more of glioma cell, GL261 cell, U373 cell and GBM5 cell.

In another preferred embodiment, the tumor comprises one or more of glioblastoma, gliomas, and astrogliomas.

In another preferred embodiment, the tumor is labeled with fluorescence.

In another preferred embodiment, the bearing site of tumor-bearing comprises one or more of intracranial and subcutaneous site.

In another preferred embodiment, the bearing site of tumor-bearing comprises intracranial brain site.

In the fourth aspect of the present invention, a tumor-bearing non-human mammalian model expressing IL-8 is provided.

In another preferred embodiment, the tumor-bearing non-human mammalian model expressing IL-8 bears tumor and express IL-8.

In another preferred embodiment, the tumor-bearing non-human mammalian model expressing IL-8 is the non-human mammalian model expressing IL-8 which bears tumor.

In another preferred embodiment, the non-human mammalian model expressing IL-8 is according to the first aspect of the present invention.

In another preferred embodiment, the tumor-bearing non-human mammalian model expressing IL-8 is heterozygous or homozygous.

In another preferred embodiment, the tumor comprises human tumor or non-human mammalian tumor.

In another preferred embodiment, the non-human mammalian is rodent.

In another preferred embodiment, the non-human mammalian comprises mice, rat, rabbit and/or monkey.

In another preferred embodiment, the mice include C57BL/6 mice.

In another preferred embodiment, the tumor comprises one or more of colon cancer, lung cancer, breast cancer, pancreatic cancer, brain tumor and leukemia.

In another preferred embodiment, the leukemia comprises acute myeloid leukemia M5.

In another preferred embodiment, the cell of the tumor comprises one or more of glioma cell, GL261 cell, U373 cell and GBM5 cell.

In another preferred embodiment, the tumor comprises one or more of glioblastoma, gliomas, and astrogliomas.

In another preferred embodiment, the tumor is labeled with fluorescence.

In another preferred embodiment, the loading site comprises one or more of intracranial and subcutaneous site.

In another preferred embodiment, the bearing site of tumor-bearing comprises intracranial brain site.

In the fifth aspect of the present invention, a method for preparing the tumor-bearing non-human mammalian model expressing IL-8 according to the fourth aspect of the present invention is provided, the method comprises:
(i) preparing the non-human mammalian model expressing IL-8;
(ii) subjecting the non-human mammalian model expressing IL-8 to bear the tumor, thereby obtaining the tumor-bearing non-human mammalian model expressing IL-8.

In another preferred embodiment, in the step (i), the preparing the non-human mammalian model expressing IL-8 is according to the second aspect of the present invention.

In another preferred embodiment, the step (ii) comprises:
inoculating the tumor cells into the non-human mammalian model expressing IL-8, thereby obtaining the tumor-bearing non-human mammalian model expressing IL-8.

In another preferred embodiment, compared with a tumor-bearing non-human mammalian model without expressing IL-8, the tumor-bearing non-human mammalian model expressing IL-8 comprises one or more features selected from the group consisting of:
(t1) expressing IL-8;
(t2) accelerating tumor growth;
(t3) increasing tumor size;
(t4) increasing tumor vascular density;
(t5) increasing the expression of IL-8 in CD4+ T cells;
(t6) increasing the expression of IL-8 in CD45+CD3− myeloid cells; and/or
(t7) carrying more exhaustion markers PD-1 and TIM-3 on tumor-infiltrating CD4+ and CD8+ T cells.

In another preferred embodiment, the IL-8 comprises serum IL-8 or tissue IL-8.

In the sixth aspect of the present invention, a use of the tumor-bearing non-human mammalian model expressing IL-8 according to the fourth aspect of the present invention for studying tumor diseases is provided.

In the seventh aspect of the present invention, a use of the tumor-bearing non-human mammalian model expressing IL-8 according to the fourth aspect of the present invention for studying the effect of IL-8 on tumor is provided.

In another preferred embodiment, the effect comprises growth effect.

In the eighth aspect of the present invention, a use of the tumor-bearing non-human mammalian model expressing IL-8 according to the fourth aspect of the present invention for screening or identifing therapeutic agents that can prevent and/or treat tumor is provided.

In another preferred embodiment, the therapeutic agent comprises drug.

In the ninth aspect of the invention, a method of screening or identifying a potential therapeutic agent for preventing and/or treating tumors, the method comprises:
(1) in a test group, in the presence of a test substance, the test substance is administered to the tumor-bearing non-human mammalian model expressing IL-8 according to the fourth aspect of the present invention, and the growth status Q1 of tumor in the tumor-bearing non-human mammalian model expressing IL-8 is measured; and in the control group in which the test substance is not administered and the other conditions are the same, the growth status Q2 of tumor in the tumor-bearing non-human mammalian model expressing IL-8 is measured;
(2) comparing the growth status Q1 and growth status Q2 measured in the step (1) to determine whether the test substance is a potential therapeutic agent for preventing and/or treating tumors;
wherein, if the growth status Q1 is lower than the growth status Q2, it indicates that the test substance is a potential therapeutic agent for preventing and/or treating tumors.

In another preferred embodiment, the growth status of the tumor comprises one or more indicators selected from the group consisting of: tumor morphological changes, tumor volume changes, tumor weight changes, and combination thereof.

In another preferred embodiment, the lower comprises significantly lower.

Preferably, the “lower” means that the growth status Q1 of the tumor in the test group having biological repeatability is lower than the growth status Q2 of the tumor in the control group having biological repeatability after administrating the test substance and the P value is less than 0.05 in a t-test.

In another preferred embodiment, the “lower” means that the ratio of growth status Q1/growth status Q2 is less than 1.0, preferably ≤0.9, more preferably ≤0.8, more preferably ≤0.7, more preferably ≤0.6, more preferably ≤0.5, more preferably ≤0.4, more preferably ≤0.3, more preferably ≤0.2, more preferably ≤0.1, more preferably ≤0.05, more preferably ≤0.01.

In another preferred embodiment, the method is non-diagnostic and non-therapeutic.

In another preferred embodiment, the test substance is selected from the group consisting of antibody, small molecule compound, nucleic acid, and combinations thereof.

In another preferred embodiment, the test substance comprises one or more of an IL-8 inhibitor and a PD-1 inhibitor.

In another preferred embodiment, the test substance comprises IL-8 inhibitor and a PD-1 inhibitor.

In another preferred embodiment, the potential therapeutic agent is selected from the group consisting of antibody, small molecule compound, nucleic acid, and combinations thereof.

In another preferred embodiment, the potential therapeutic agent comprises one or more of an IL-8 inhibitor and a PD-1 inhibitor.

In another preferred embodiment, the inhibitor is selected from the group consisting of antibody, small molecule compound, nucleic acid, and combinations thereof.

In another preferred embodiment, the IL-8 inhibitor comprises anti-IL-8 antibody.

In another preferred embodiment, the PD-1 inhibitor comprises anti-PD-1 antibody.

In another preferred embodiment, the inhibitor comprises a specific inhibitor or a non-specific inhibitor.

In the tenth aspect of the present invention, a pharmaceutical composition comprising an IL-8 inhibitor and a PD-1 inhibitor; and a pharmaceutically acceptable carrier is provided.

In another preferred embodiment, the formulation of the pharmaceutical composition is solid preparation, liquid preparation or semi-solid preparation.

In another preferred embodiment, the formulation of the pharmaceutical composition is oral preparation, external preparation or injection preparation.

In another preferred embodiment, the injection preparation is intravenous injection preparation.

In another preferred embodiment, the formulation of the pharmaceutical composition is tablet, injection, infusion, ointment, gel, solution, microsphere or film.

In another preferred embodiment, the content of the IL-8 inhibitor is 0.001-99.9 wt %, based on the weight of the pharmaceutical composition.

In another preferred embodiment, the content of the PD-1 inhibitor is 0.001-99.9 wt %, based on the weight of the pharmaceutical composition.

In the eleventh aspect of the present invention, a use of the pharmaceutical composition according to the tenth aspect of the present invention for preparing a medicament for preventing and/or treating tumor is provided.

In another preferred embodiment, the tumor comprises human tumor or non-human mammalian tumor.

In another preferred embodiment, the non-human mammalian is rodent.

In another preferred embodiment, the non-human mammalian comprises mice, rat, rabbit and/or monkey.

In another preferred embodiment, the mice include C57BL/6 mice.

In another preferred embodiment, the tumor comprises one or more of colon cancer, lung cancer, breast cancer, pancreatic cancer, brain tumor and leukemia.

In another preferred embodiment, the leukemia comprises acute myeloid leukemia M5.

In another preferred embodiment, the cell of the tumor comprises one or more of glioma cell, GL261 cell, U373 cell and GBM5 cell.

In another preferred embodiment, the tumor comprises one or more of glioblastoma, gliomas, and astrogliomas.

In another preferred embodiment, the tumor is labeled with fluorescence.

In the twelfth aspect of the present invention, a method for preventing and/or treating tumors is provided, the method comprises: administering the pharmaceutical composition according to the tenth aspect of the present invention to a subject in need is provided.

In another preferred embodiment, the subject comprises human or non-human mammalian.

In another preferred embodiment, the non-human mammalian comprises rodent, rabbit, monkey, livestock, dog, cat, etc.

In another preferred embodiment, the administration is injection administration or oral administration.

In another preferred embodiment, the injection administration is intravenous injection administration.

In the thirteenth aspect of the present invention, a use of an IL-8 inhibitor for preparing a pharmaceutical composition for enhancing anti-tumor effect of PD-1 inhibitor.

In another preferred embodiment, the pharmaceutical composition further comprises a pharmaceutically acceptable carrier.

In another preferred embodiment, the formulation of the pharmaceutical composition is solid preparation, liquid preparation or semi-solid preparation.

In another preferred embodiment, the formulation of the pharmaceutical composition is oral preparation, external preparation or injection preparation.

In another preferred embodiment, the injection preparation is intravenous injection preparation.

In another preferred embodiment, the formulation of the composition is tablet, injection, infusion, ointment, gel, solution, microsphere or film.

In another preferred embodiment, the tumor comprises human tumor or non-human mammalian tumor.

In another preferred embodiment, the non-human mammal is rodent.

In another preferred embodiment, the non-human mammalian comprises mice, rat, rabbit and/or monkey.

In another preferred embodiment, the mice comprises C57BL/6 mice.

In another preferred embodiment, the tumor comprises one or more of colon cancer, lung cancer, breast cancer, pancreatic cancer, brain tumor and leukemia.

In another preferred embodiment, the leukemia comprises acute myeloid leukemia M5.

In another preferred embodiment, the cell of the tumor comprises one or more of glioma cell, GL261 cell, U373 cell and GBM5 cell.

In another preferred embodiment, the tumor comprises one or more of glioblastoma, gliomas, and astrogliomas.

It should be understood that, within the scope of the present invention, each technical feature of the present invention described above and in the following (as examples) may be combined with each other to form a new or preferred technical solution, which is not listed here due to space limitations.

### DESCRIPTION OF THE DRAWINGS

FIG. 1 shows generation of humanized mouse carrying human IL8 gene locus (IL8-Hu). (1A) shows human BAC clone (PiggyBac-on-BAC RP11-997L11) comprising a human IL8 gene containing the entire human IL8 gene locus and upstream/downstream regulatory elements was inserted into the C57BL/6 mouse genome. Four pairs of BAC-specific primers that amplified 300-800 bp fragments from different parts of the BAC plasmid were used to identify BAC-positive pups by PCR analysis. Offspring carrying the intact human IL8 gene locus were back-crossed with C57BL/6 mice for 5 generations; (1B) shows the IL8 insertion site in mouse genome. Inverse PCR was used to identify the IL8 insertion site as previously described. Briefly, genomic DNA was extracted from tails, digested with HaeIII (NEB, Cat #R0108S), and then ligated with T4 DNA Ligase (NEB, Cat #M0202L). The primers used to recover the flanking sequence of the left side of the PiggyBac transposon were PB-5IF (5′-CTTGACCTTGCCACAGAGGACTATTAGAGG-3′ (SEQ ID NO: 1)) and PB-5IR (5′-CAGTGACAC TTACCGCATTGACAAGCACGC-3′ (SEQ ID NO: 2)). The primers used to recover the flanking sequence of the right side of the piggyBac transposon were PB-3IF (5′-CCTCGATATACAGACCGATAAAACACATGC-3′ (SEQ ID NO: 3)) and PB-3IR (5′-AGTCAGTCA GAAACAACTTTGGCACATATC-3′ (SEQ ID NO: 4)). PCR products were cloned into pMD19-T (Sino Biological) for subsequent sequencing. Sequencing results were analyzed with NCBI BLAST searches and Ensemble human and mouse genome databases. Human IL8 gene locus was inserted at 3023490-3024411 position of mouse chromosome 9.

FIG. 2 shows immune cell percentage of IL8-Hu mouse. (2A), (2B), (2C) show flow cytometry analysis of immune cell percentages in IL8-Hu mice. Macrophages, CD11b+ F4/80+; granulocytes, CD11b+ Ly6G+; Treg, CD4+ Foxp3+; B cells, B220+; conventional dendritic cells (cDCs), MHCII+ CD11c+; spleen cDC1, MHCII+ CD11c+ CD8+; spleen cDC2, MHCII+ CD11c+ CD11b+; naïve T cells (Tn), CD62L+ CD44−; effector T cells (Teff), CD62L− CD44+; central memory T cells (Tcm), CD62L+ CD44+; immature cDCs, I-A/I-Emid CD11c+; mature cDCs, I-A/I-Ehi CD11c+. SP, single-positive cells; DP, CD4 and CD8 double-positive cells.

FIG. 3 shows tumor growth in IL8-Hu mouse. (3A) shows in vivo bioluminescent imaging of tumor growth. A total of 10,000 GL261-Luc cells were implanted into WT or IL8-Hu mouse brains. Twenty-five days later, tumor growth was measured. The scale indicates luminescence strength as p/s/cm2/sr. (3B) shows survival curve of WT and IL8-Hu mice bearing GL261 tumors. A total of 10,000 GL261-Luc cells were injected into the callosum and monitored for survival. WT, n=8, IL8-Hu, n=7. ***P<0.001, log-rank test. (3C) shows flow cytometry analysis of IL-8-producing cells in GL261 tumors of IL8-Hu mice. WT or IL8-Hu mice were implanted with GL261 tumors as described in C. On day 20, the mice were sacrificed. Percoll-enriched cells were stimulated with PMA/ionomycin in the presence of GolgiPlug and stained for flow cytometry analysis. The red color indicates CXCR5+ CD4+ T cells. Graph data are displayed as the mean±SD. ****P<0.0001, two-tailed t test. (3D) shows CD31 IHC staining of GL261 tumors. The CD31+ area per view was analyzed and quantified by ImageJ software. Graph data are displayed as the mean±SD. ****P<0.0001, two-tailed t test.

FIG. 4 shows T cells in IL8-Hu mouse tumor have a more exhausted status. (4A) and (4B) show flow cytometry analysis of PD-1 and TIM-3 expression on T cells. Graph data are displayed as the mean±SD. *P<0.05, **P<0.01, two-tailed t test.

FIG. 5 shows systemic elevation of IL-8 expression by anti-PD-1 treatment. (5A) In vivo bioluminescent imaging of tumor growth. A total of 10,000 GL261-Luc-IL8 cells were implanted into the IL8-Hu mouse brain. From day 7, mice were intraperitoneally injected with 200 μg/mouse anti-PD-1 (RMP1-14) antibody or isotype control antibody every three days for four times in total. On day 26, tumor growth was imaged. The scale indicates luminescence strength as p/s/cm2/sr. (5B) Flow cytometry analysis of PD-1 and TIM-3 expression on tumor T cells. Graph data are displayed as the mean±SD. **P<0.01, two-tailed t test. (5C) IL-8 IHC staining of IL8-Hu mice bearing GL261-Luc tumors with or without anti-PD-1 treatment. A total of 10,000 tumor cells were implanted into the IL8-Hu mouse brain. On day 18, 200 μg/mouse of anti-PD-1 antibody was intraperitoneally injected. Five days later, IL-8 expression was measured by IHC staining. The IL-8+ spot number and IL-8+ area per view were analyzed using ImageJ software. Graph data are displayed as the mean±SD. ****P<0.0001, two-tailed t test. (5D) Serum was collected 5 days after antibody treatment, and IL-8 level was measured by ELISA. Graph data are displayed as the mean±SD. ***P<0.001, two-tailed t test. (5E) Flow cytometry analysis of tumor MDSCs. Immune cells from naïve WT or IL8-Hu mouse brains (upper) or tumor-bearing IL8-Hu mice treated with anti-PD-1 antibody or isotype (lower) were enriched by Percoll density gradient centrifugation and stained for flow cytometry. The statistical analysis is shown on the right. Graph data are displayed as the mean±SD. *P<0.05, **P<0.01, two-tailed t test. (5F) Representative pattern of Ly6C and Ly6G staining in indicated tissues. (5G) Flow cytometry analysis of MDSCs from the indicated tissues of tumor-bearing mice with or without anti-PD-1 treatment. The statistical analysis is shown below. Graph data are displayed as the mean±SD. *P<0.05, **P<0.01, ***P<0.001, two-tailed t test.

FIG. 6 shows blockade of the IL-8-CXCR1/CXCR2 axis potentiates anti-PD-1 efficacy; (6A) In vivo bioluminescent imaging of tumor growth. A total of 20,000 GL261-Luc cells were implanted into WT or IL8-Hu mouse brains. From day 7, mice were intraperitoneally injected with 200 μg/mouse anti-PD-1 (clone RMP1-14) antibody or isotype IgG every three days for four times in total. At the same time, a portion of the mice were given 50 μg reparixin by intraperitoneal injection every other day until sacrifice. On day 20, tumor growth was imaged. The scale indicates luminescence strength as p/s/cm2/sr; (6B) Survival curve of mice with different treatment described as in A. *P<0.05, **P<0.01, ***P<0.001, log-rank test. (6C) Flow cytometry analysis of Gr1 and CD11b expressing cells in tumor on day 21 after tumor injection. (6D) Statistical analysis of the percentage and cell number of CD11bhi Gr1mid, CD11bhi Gr1hi and CD11bhi Gr1-cells in tumors. The 95% CIs are shown in translucent shadows (brown for WT mice, cyan for IL8-Hu mice). Brown stars show the corresponding P value compared between WT groups and cyan stars show the P value compared between IL8-Hu groups. The dark stars show the corresponding P value between WT and IL8-Hu groups. Graph data are displayed as the mean±SD. *P<0.05, **P<0.01, ***P<0.001, ****P<0.0001, two-tailed t test.

FIG. 7 shows neutralize IL-8 potentiates anti-PD-1 efficacy. (7A) Schematic diagram of the experimental approach. Five hundred GL261-Luc-IL8 cells were implanted into IL8-Hu mouse brains. From day 7, mice were given 200 μg anti-PD-1 antibody (clone RMP1-14) or 200 μg anti-IL-8 antibody (clone 6217) per mouse, intraperitoneally. On day 26, tumor growth was imaged. (7B) Mice were treated as shown in F. In vivo bioluminescent imaging of tumor growth at day 26. The scale indicates luminescence strength as p/s/cm2/sr. (7C) Survival curve of the mice with different treatments as in F. ****P<0.0001, log-rank test. (7D). IHC staining of tumor tissue with antibody against CD31. The CD31+ area (vascular area) per view was counted using ImageJ software. Graph data are displayed as the mean±SD. *P<0.05, ***P<0.001, ****P<0.0001, two-tailed t test.

### EMBODIMENTS FOR CARRYING OUT THE INVENTION

The present invention prepare a non-human mammalian model expressing IL-8, the non-human mammalian model expressing IL-8 can be better used to prepare a tumor-bearing non-human mammalian model expressing IL-8 which can be used for studying the effect of IL-8 on tumors and screening or identifying potential therapeutic agents for the prevention and/or treatment of tumors. The present invention also provides the use of an IL-8 inhibitor for enhancing the anti-tumor effect of a PD-1 inhibitor.

### Terms

As used herein, the terms “comprise”, “comprising”, and “containing” are used interchangeably, which not only comprise closed definitions, but also semi-closed and open definitions. In other words, the term comprises “consisting of” and “essentially consisting of”.

As used herein, the terms “interleukin 8” is abbreviated as IL-8.

As used herein, the terms “PD-1” refers to “programmed death-1”, the “programmed death-1” and “programmed cell death protein 1” can be used interchangeably.

### IL-8

In the present invention, IL-8 refers to interleukin-8.

In a preferred embodiment of the present invention, the IL-8 comprises human IL-8 or primate IL-8.

In another preferred embodiment, the IL-8 comprises serum IL-8 or tissue IL-8. It is unexpectedly found in the present invention that IL-8 inhibitors can significantly enhance the anti-tumor effect of PD-1 inhibitor, and the combined administration of PD-1 inhibitor and IL-8 inhibitor can significantly improve the anti-tumor effect.

### Non-Human Mammalian

In the present invention, the non-human mammalian can be rodent.

In a preferred embodiment, the non-human mammalian comprises mice, rat, rabbit and/or monkey.

In another preferred embodiment, the mice comprises C57BL/6 mice.

### Tumor

In the present invention, the tumor can be human tumor or non-human mammalian tumor.

In a preferred embodiment, the tumor comprises one or more of colon cancer, lung cancer, breast cancer, pancreatic cancer, brain tumor and leukemia.

Typically, the leukemia comprises acute myeloid leukemia M5.

In a preferred embodiment, the cell of the tumor comprises one or more of glioma cell, GL261 cell, U373 cell and GBM5 cell.

In another preferred embodiment, the tumor comprises one or more of glioblastoma, gliomas, and astrogliomas.

In another preferred embodiment, the tumor is labeled with fluorescence.

### Non-Human Mammalian Model Expressing IL-8, Preparing Method and Use Thereof

The present invention provide a non-human mammalian model expressing IL-8.

In a preferred embodiment, the expression of IL-8 comprises serum expression of IL-8 or tissue expression of IL-8.

The present invention provide a method for preparing the non-human mammalian model expressing IL-8 of the present invention, the method comprises:
subjecting the non-human mammalian to express IL-8, thereby obtaining the non-human mammalian model expressing IL-8.

In a preferred embodiment, the method for preparing the non-human mammalian model expressing IL-8 comprises:
subjecting the non-human mammalian to having a gene expressing IL-8, thereby obtaining the non-human mammalian model expressing IL-8.

In another preferred embodiment, the method for preparing the non-human mammalian model expressing IL-8 comprises:
inserting a gene expressing IL-8 into the non-human mammalian to make the non-human mammalian express IL-8, thereby obtaining the non-human mammalian model expressing IL-8.

In another preferred embodiment, the gene expressing IL-8 is located in the genome of the non-human mammalian.

In another preferred embodiment, the gene expressing IL-8 is located at positions 3023490-3024411 of chromosome 9 of the non-human mammalian cells.

In another preferred embodiment, the method for preparing the non-human mammalian model expressing IL-8 comprises:
(a) providing the non-human mammalian cell, subjecting the cell to express IL-8, thereby obtaining the non-human mammalian cell expressing IL-8;
(b) using the non-human mammalian cell expressing IL-8 obtained in step (a) to prepare the non-human mammalian model expressing IL-8.

In another preferred embodiment, in step (a), the genome of the cell comprises a gene expressing IL-8.

In another preferred embodiment, the step (a) comprises:
providing the non-human mammalian cell, a gene expressing IL-8 was inserted into the cell, thereby obtaining the non-human mammalian cell expressing IL-8;

In another preferred embodiment, the step (a) comprises:
inserting the gene expressing IL-8 into the genome of the non-human mammalian cell by using gene insertion technology to obtain the non-human mammalian cell expressing IL-8.

In another preferred embodiment, the step (b) comprises:
(b1) subjecting the non-human mammalian cell expressing IL-8 obtained in step (a) to prepare a chimeric non-human mammalian;
(b2) mating the chimeric non-human mammal obtained in step (b1) with a normal wild-type non-human mammalian, and screening progeny to obtain a heterozygous non-human mammalians expressing IL-8;
(b3) mating the heterozygous non-human mammalians obtained in step (b2) with each other to obtain the non-human mammal model expressing IL-8.

The present invention provide a use of the non-human mammalian model expressing IL-8 of the present invention for preparing a tumor-bearing non-human mammalian model expressing IL-8.

### Tumor-Bearing Non-Human Mammalian Model Expressing IL-8, Preparing Method and Use Thereof

The present invention provide a tumor-bearing non-human mammalian model expressing IL-8.

In another preferred embodiment, the tumor-bearing non-human mammalian model expressing IL-8 is the non-human mammalian model expressing IL-8 which bears tumor.

In another preferred embodiment, the bearing site of tumor-bearing comprises one or more of intracranial and subcutaneous site.

Typically, the bearing site of tumor-bearing comprises intracranial brain site.

The present invention provide a method for preparing the tumor-bearing non-human mammalian model expressing IL-8 of the present invention is provided, the method comprises:
(i) preparing the non-human mammalian model expressing IL-8;
(ii) subjecting the non-human mammalian model expressing IL-8 to bear the tumor, thereby obtaining the tumor-bearing non-human mammalian model expressing IL-8.

In another preferred embodiment, the step (ii) comprises:
inoculating the tumor cells into the non-human mammalian model expressing IL-8, thereby obtaining the tumor-bearing non-human mammalian model expressing IL-8.

In another preferred embodiment, compared with a tumor-bearing non-human mammalian model without expressing IL-8, the tumor-bearing non-human mammalian model expressing IL-8 comprises one or more features selected from the group consisting of:
(t1) expressing IL-8;
(t2) accelerating tumor growth;
(t3) increasing tumor size;
(t4) increasing tumor vascular density;
(t5) increasing the expression of IL-8 in CD4+ T cells;
(16) increasing the expression of IL-8 in CD45+CD3− myeloid cells; and/or
(t7) carrying more exhaustion markers PD-1 and TIM-3 on tumor-infiltrating CD4+ and CD8+ T cells.

In another preferred embodiment, the expression of IL-8 comprises serum expression or tissue expression.

The present invention provide a use of the tumor-bearing non-human mammalian model expressing IL-8 of the present invention for studying tumor diseases.

The present invention provide a use of the tumor-bearing non-human mammalian model expressing IL-8 of the present invention for studying the effect of IL-8 on tumor is provided.

In another preferred embodiment, the effect comprises growth effect.

The present invention provide a use of the tumor-bearing non-human mammalian model expressing IL-8 of the present invention for screening or identifing therapeutic agents that can prevent and/or treat tumor is provided.

In another preferred embodiment, the therapeutic agent comprises drug.

### Screening or Identifying a Potential Therapeutic Agent for Preventing and/or Treating Tumors

The present application provides a method of screening or identifying a potential therapeutic agent for preventing and/or treating tumors, the method comprises:
(1) in a test group, in the presence of a test substance, the test substance is administered to the tumor-bearing non-human mammalian model expressing IL-8 according to the fourth aspect of the present invention, and the growth status Q1 of tumor in the tumor-bearing non-human mammalian model expressing IL-8 is measured; and in the control group in which the test substance is not administered and the other conditions are the same, the growth status Q2 of tumor in the tumor-bearing non-human mammalian model expressing IL-8 is measured;
(2) comparing the growth status Q1 and growth status Q2 measured in the step (1) to determine whether the test substance is a potential therapeutic agent for preventing and/or treating tumors;
wherein, if the growth status Q1 is lower than the growth status Q2, it indicates that the test substance is a potential therapeutic agent for preventing and/or treating tumors.

In a preferred embodiment, the growth status of the tumor comprises one or more indicators selected from the group consisting of: tumor morphological changes, tumor volume changes, tumor weight changes, and combination thereof.

In another preferred embodiment, the lower comprises significantly lower.

Preferably, the “lower” means that the growth status Q1 of the tumor in the test group having biological repeatability is lower than the growth status Q2 of the tumor in the control group having biological repeatability after administrating the test substance and the P value is less than 0.05 in a t-test.

Preferably, the “lower” means that the ratio of growth status Q1/growth status Q2 is less than 1.0, preferably ≤0.9, more preferably <0.8, more preferably ≤0.7, more preferably ≤0.6, more preferably ≤0.5, more preferably ≤0.4, more preferably ≤0.3, more preferably ≤0.2, more preferably ≤0.1, more preferably ≤0.05, more preferably ≤0.01.

In another preferred embodiment, the method is non-diagnostic and non-therapeutic.

In another preferred embodiment, the test substance is selected from the group consisting of antibody, small molecule compound, nucleic acid, and combinations thereof.

In another preferred embodiment, the test substance comprises one or more of an IL-8 inhibitor and a PD-1 inhibitor.

In another preferred embodiment, the potential therapeutic agent is selected from the group consisting of antibody, small molecule compound, nucleic acid, and combinations thereof.

In another preferred embodiment, the potential therapeutic agent comprises one or more of IL-8 inhibitor and PD-1 inhibitor.

In another preferred embodiment, the inhibitor is selected from the group consisting of antibody, small molecule compound, nucleic acid, and combinations thereof.

In another preferred embodiment, the IL-8 inhibitor comprises anti-IL-8 antibody.

In another preferred embodiment, the PD-1 inhibitor comprises anti-PD-1 antibody.

In another preferred embodiment, the inhibitor comprises a specific inhibitor or a non-specific inhibitor.

### Pharmaceutical Composition

Typically, the composition is a pharmaceutical composition.

In a preferred embodiment, the pharmaceutical composition further comprises a pharmaceutically acceptable carrier

In another preferred embodiment, the formulation of the pharmaceutical composition or preparation is solid preparation, liquid preparation or semi-solid preparation.

In another preferred embodiment, the formulation of the pharmaceutical composition is oral preparation, external preparation or injection preparation.

In another preferred embodiment, the injection preparation is intravenous injection preparation.

In another preferred embodiment, the formulation of the pharmaceutical composition is tablet, injection, infusion, ointment, gel, solution, microsphere or film.

The term “pharmaceutically acceptable carrier” refers to one or more compatible solid, semi-solid, liquid or gel fillers, which are suitable for use in humans or animals and must have sufficient purity and sufficient low toxicity. The “compatible” means each ingredient of the pharmaceutical composition and active ingredient of the drug can be blended with each other without significantly reducing the efficacy.

It should be understood that the carrier is not particularly limited. In the present invention, the carrier can be selected from materials commonly used in the art, or can be obtained by a conventional method, or is commercially available. Some examples of pharmaceutically acceptable carriers are cellulose and its derivatives (such as methylcellulose, ethylcellulose, hydroxypropylmethylcellulose, sodium carboxymethylcellulose, etc.), gelatin, talc, solid lubricants (such as stearic acid, magnesium stearate), calcium sulfate, plant oil (such as soybean oil, sesame oil, peanut oil, olive oil, etc.), polyols (such as propylene glycol, glycerin, mannitol, sorbitol, etc.), emulsifier (such as Tween), wetting agent (such as sodium lauryl sulfate), buffer agent, chelating agent, thickener, pH regulator, transdermal enhancer, colorant, flavoring agent, stabilizer, antioxidant, preservative, bacteriostatic agent, pyrogen-free water, etc.

Representatively, in addition to the active pharmaceutical ingredient, the liquid formulations can contain inert diluents commonly used in the art, such as water or other solvents, solubilizers and emulsifiers, for example, ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil or a mixture thereof. In addition to these inert diluents, the composition can also contain adjuvants such as wetting agents, emulsifiers and suspensions and the like.

The pharmaceutical preparation should be matched with the mode of administration. The formulation of present invention can also be used together with other synergistic therapeutic agents (including before, simultaneous or after administering). When a pharmaceutical composition or preparation is administered, a safe and effective dose of drug is administered to a subject in need (e.g. human or non-human mammals). The safe and effective dose is usually at least 10 μg/kg body weight, and does not exceed about 8 mg/kg body weight in most cases, and preferably the dose is about 10 μg/kg body weight to about 1 mg/kg body weight. Of course, the route of administration, patient health and other factors, should also be taken into account to determine the specific dose, which are within the ability of the skilled physicians.

### The Main Advantages of the Present Invention Comprises

1. The present invention prepare a non-human mammalian model expressing IL-8, the non-human mammalian model expressing IL-8 can be better used to prepare a tumor-bearing non-human mammalian model expressing IL-8 which can be used for studying the effect of IL-8 on tumors and screening or identifying potential therapeutic agents for the prevention and/or treatment of tumors.
2. The present invention also provides the use of an IL-8 inhibitor for enhancing the anti-tumor effect of a PD-1 inhibitor. The combined administration of PD-1 inhibitor and an IL-8 inhibitor can significantly improve the anti-tumor effect.

The present invention will be further illustrated below with reference to the specific examples. It should be understood that these examples are only to illustrate the invention but not to limit the scope of the invention. The experimental methods with no specific conditions described in the following examples are generally performed under the conventional conditions, or according to the manufacturer's instructions.

### EXAMPLE 1

### 1. Method

### IL-8 Humanized (IL8-Hu) Mouse Strain Generation

PiggyBac-on-BAC RP11-997L11, which contains the human IL8 locus, was inserted into the C57BL/6 mouse genome. Four pairs of BAC-specific primers that amplified 300-800 bp fragments from different parts of the BAC plasmid were used to identify BAC-positive pups by PCR analysis. Offspring carrying the intact human IL8 gene locus were back-crossed with C57BL/6 mice for 5 generations. Inverse PCR was used to identify the IL8 insertion site. Briefly, genomic DNA was extracted from tails, digested with HaeIII (NEB, Cat #R0108S), and then ligated with T4 DNA Ligase (NEB, Cat #M0202L). The primers used to recover the flanking sequence of the left side of the Piggy Bac transposon were PB-5IF (5′-CTTGACCTTGCCACAGAGGACTATTAGAGG-3′ (SEQ ID NO: 1)) and PB-5IR (5′-CAGTGACAC TTACCGCATTGACAAGCACGC-3′ (SEQ ID NO: 2)). The primers used to recover the flanking sequence of the right side of the piggyBac transposon were PB-3IF (5′-CCTCGATATACAGACCGATAAAACACATGC-3′ (SEQ ID NO: 3)) and PB-3IR (5′-AGTCAGTCA GAAACAACTTTGGCACATATC-3′ (SEQ ID NO: 4)). PCR products were cloned into pMD19-T (Sino Biological) for subsequent sequencing. Sequencing results were analyzed with NCBI BLAST searches (www.ncbi.nlm.nih.gov) and Ensemble human and mouse genome databases (www.ensembl.org). Human IL8 gene locus was inserted at 3023490-3024411 position of mouse chromosome 9. All mice used in this study were kept in individually ventilated cages under specific-pathogen-free conditions, and all mouse protocols were approved by the ethics committees of TMMU.

### Glioma Cell Lines

GL261 (ATCC, Cat #CRL-1887) and U373 MG cells (European Collection of Authenticated Cell Cultures (ECACC), Cat #08061901) were transfected with lentivirus carrying Luc-GFP, and GFP-positive cells were sorted by flow cytometry to generate a luciferase-stable cell line. To establish an IL-8-producing cell line, IL8 was overexpressed in GL261-Luc cells using lentivirus carrying IL8-RFP, and RFP-positive cells were sorted by flow cytometry. Cells were maintained in vitro with DMEM supplemented with 10% fetal bovine serum (FBS) (Gibco, Cat #10099), 2 mM GlutaMAX, and 100 U/mL penicillin/streptomycin and cultured at 37° C. and 5% CO₂.

### Glioma Mouse Model

For the GL261 tumor model, C57BL/6 or IL8-Hu mice (8-12 weeks old; 3-10 mice per group) were anesthetized using a mixture of ketamine (50 mg/kg) and xylazine (5 mg/kg) injected intraperitoneally. Mouse heads were shaved and then placed in a stereotaxic frame. After the sterilization of the scalp with alcohol and betadine, a 1-centimeter midline scalp incision was made to expose the skull. Then, 4% H₂O₂ was used to remove the periosteum, and a burr hole was drilled 2 mm lateral and 1 mm posterior from the bregma. A 10 μL syringe (Hamilton, 1701 RN no NDL) with a 33 G needle was injected at a depth of 2 millimeters (mm) and retracted 0.5 mm to form a reservoir. Using a microinfusion pump, tumor cells were injected in a volume of 3 μL at a speed of 1 μL/minute. The syringe was left in place for 1 minute before removal of the syringe. Bone wax was used to fill the burr hole, and the skin was conglutinated and cleaned. Mice were placed in a heated cage until full recovery. GBM5, U373-Luc, or U373-Luc plus T cells were injected into 6-8-week-old B-NDG mice (NOD. CB17-Prkdc^{scid}Il2rg^{tm1}/Bcgen, Biocytogen, China) as described above, with 5-7 mice per group. To measure tumor growth, tumor-bearing mice were anesthetized using isoflurane intraperitoneally and injected intraperitoneally with D-Luciferin (150 mg/kg body weight; Promega, Cat #P1043). After 10 minutes, the animals were transferred to the IVIS 100 imaging system (Caliper Life Sciences), and luminescence was detected. Data were subsequently analyzed using Living Image 2.5 software (Caliper Life Sciences).

Preparation of Cell Suspensions from Mouse Tumor Tissues.

Tumor tissue was cut into pieces and incubated in C-Tubes (Miltenyi Biotec, Cat #130-096-334) with digestion cocktail containing 1 mg/mL collagenase D (Roche, Cat #11088882001) and 30 μg/mL DNase I (Sigma-Aldrich, Cat #D7291-2 MG) in complete RPMI 1640 medium (10% FBS) at 37° C. for 15 minutes and then dissociated on gentleMACS with Program m_brain_01 followed by incubation for another 15 minutes at 37° C. Afterward, the homogenate was filtered through a 70 μm strainer and centrifuged at 500× g for 10 minutes at 4° C. This was followed by an immune cell enrichment step using density gradient centrifugation with 30% and 70% Percoll (Fisher Scientific, Cat #10607095) in RPMI 1640 medium containing 10% FBS. After centrifuge at 2000× g for 20 minutes at 20° C. (without brakes during acceleration and deceleration), the middle transparent layer containing immune cells was collected. After washing with PBS containing 1.5% FBS and 20 mM EDTA, the cells were ready for flow cytometry analysis or sorting.

### Cell Surface and Intracellular Cytokine Staining for Flow Cytometry

Cells from various experiments were stained with a fluorescence-labeled antibody cocktail for 30 minutes at 4° C. and then analyzed with a BD LSRFortessa instrument. To measure cytokines in lymphocytes, cells were stimulated with 100 ng/mL phorbol-12-myristate-13-acetate (PMA) and 1 μg/mL ionomycin in the presence of GolgiPlug (BD Biosciences, Cat #555028) for 5 hours. Intracellular staining was carried out with a Fixation/Permeabilization Kit (BD Biosciences, Cat #555028). Data were analyzed with FlowJo.

### Immunohistochemistry (IHC) Staining

Formalin-fixed paraffin-embedded tumor tissue blocks, or frozen tumor tissues were cut into 3 μm-thick serial sections. After blocking with streptavidin peroxidase, heat-induced antigen epitope retrieval in citrate buffer (pH: 6.0) was performed. Sections were incubated overnight at 4° C. with primary antibodies against Cit-H3 (Abcam, Cat #ab5103, 1:300 dilution), CD31 (Abcam, Cat #ab28364, 1:200 dilution), IL-8 (Bio-Rad, Cat #AHP781, 1:1000 dilution) or human CD4 (Abcam, Cat #ab133616, 1:200 dilution) followed by counterstaining with hematoxylin. Staining was visualized by a Dako REAL™ EnVision™ Detection System (Dako, Cat #K5007), and sections were scanned with an Axio Scan.Z1 (ZEISS). Quantitative analysis of CD31- and IL-8-positive regions or IL-8-positive spots was performed using ImageJ 1.46r as previously described. The images were converted to RGB stacks, and thresholding was used to measure the pixel area or particles of CD31 and IL-8 staining by a blinded observer. In this context, 3-10 randomly selected visual fields (200× magnification) of each tumor were analyzed.

### ELISA

A 96-well plate (Thermo, Cat #442404) was coated with 5 μg/mL IL-8 capture antibody (Biolegend, Cat #514602) at 4° C. overnight. After blocking with 1% BSA for 1 hour, mouse serum or standard substance (Biolegend, Cat #570909) was incubated at 4° C. overnight. After washing, 2 μg/mL biotin-labeled detection antibody (Biolegend, Cat #514704) was added and incubated at room temperature for 1 hour. Following another one-hour incubation with streptavidin-HRP (R&D, Cat #DY998), the IL-8 concentration was quantified with HRP-catalyzed oxidation of o-phenylenediamine (Sigma, Cat #P8936) and measured by a MULTISKAN GO instrument (Thermo Scientific).

Statistics. Comparisons between two different groups were performed with unpaired two-tailed Student's t tests. Survival analysis was performed by the Kaplan-Meier method, with the log-rank test for comparison. HRs and their 95% CIs were calculated using stratified Cox proportional hazards regression models. The correlation P values were calculated using a stratified two-tailed test. The cutoff point optimization of patients in the TCGA database was calculated by SPSS statistics software. Statistical analysis was performed with GraphPad Prism 9. All quantitative data are presented as the mean±SD. All P values below 0.05 were considered significant (*P<0.05; **P<0.01; ***P<0.001; ****P<0.0001).

### 2. Result

Malignant glioma has a very poor prognosis and remains challenging due to the limited options of standard treatment, including surgery and radio-/chemotherapy. Although immunotherapies targeting the inhibitory receptors PD-1 and CTLA-4 can re-energize tumor-specific T cells and produce lasting antitumor responses in various cancer types, the clinical benefit of ICB therapies for malignant glioma remains compromised. A recent phase III clinical trial of anti-PD-1 therapy failed to demonstrate a survival benefit in recurrent glioblastoma (GBM), the most aggressive type of glioma. Potential reasons for this include the intrinsic properties of glioma cells, such as their vast genetic heterogeneity but low mutation burdens, and alternatively the extrinsic feature of a highly immunosuppressive microenvironment governed by prevalent tumor-associated myeloid cells as well as multivesicular niches that sabotage T-cell-mediated antitumor immunity.

Accumulating evidence indicates that the heterogeneity and functional status of tumor-infiltrating T cells play pivotal roles in shaping antitumor immunity as well as clinical properties such as the response to immunotherapy. The glioma microenvironment harbors both CD4⁺ and CD8⁺ T cells, although less frequently than myeloid cells, and they increase paradoxically with tumor grade, implying their functional diversity. Indeed, recent efforts focusing on T cell exhaustion assessment at the single-cell level have revealed that glioma-infiltrating T cells comprise variable subpopulations, among which less than 20% of T cells are clonally expanded and categorized mainly into exhausted, effector or memory subsets, while the remaining 80-90% of tumor-infiltrating T cells, so-called bystander T cells, are not clonally expanded and their function remains unexplored. Additionally, the ontogenetic origin of T cells in glioma is unknown. Here, we conducted landscape mapping of T cells from paired human blood and tumor tissues with adaptations of single-cell RNA sequencing (scRNA-seq), CyTOF, and flow cytometry. Comparison analysis enabled us to uncover the unique features of tumor-infiltrating T cells compared with their peripheral counterparts. Specifically, we identified a distinctive subset of IL-8-producing CD4⁺ T cells that exhibits innate-like features and correlates with poor prognosis. Given that IL-8 is absent in rodents, thus circumventing the incisive investigation of IL-8-mediated immune responses in vivo and its role in blunting ICB therapy, we developed an IL-8-humanized mouse strain that carries a bacterial artificial chromosome (BAC) encompassing the entire human IL8 gene locus and upstream/downstream regulatory elements by adapting a strategy shown previously. In a glioma-transplanted mouse model, IL-8⁺CD4⁺ T cells can function alone and/or together with IL-8-producing myeloid and stromal cells to solidify the immunosuppressive properties of the glioma microenvironment by promoting angiogenesis and MDSC recruitment. Strikingly, anti-PD-1 immunotherapy elevates systemic IL-8 expression and blunts ICB efficacy by reinforcing immunosuppression. Thus, our study leverages the understanding of bystander T cells in tumors and highlights the IL-8-CXCR1/CXCR2 axis as a combinational immunotherapy target.

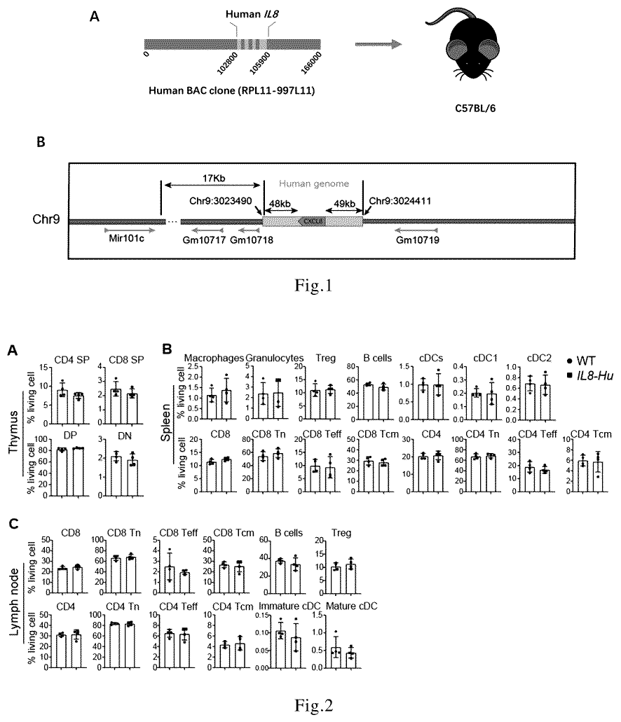

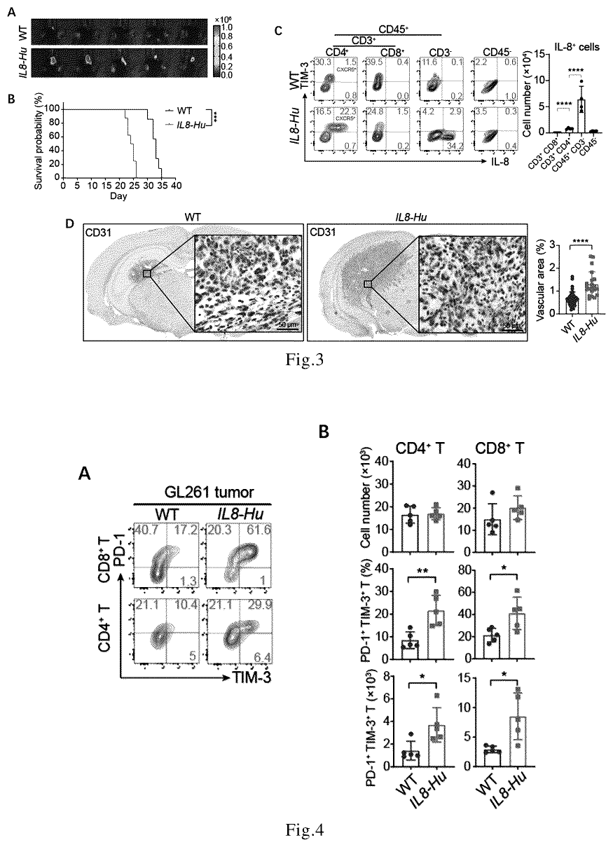

Because the IL8 gene is lacking in rodents, the in vivo function of IL-8, especially as a key immune regulator in tumorigenesis, remains unclear. We followed the previous strategy and generated an IL-8-humanized (IL8-Hu) mouse strain, in which an ˜160 kbs human BAC clone (RPL11-997L11) containing the entire human IL8 gene locus and upstream/downstream regulatory elements was inserted into the intergenic region of mouse chromosome 9 (FIG. 1A and 1B in FIG. 1). Naïve IL8-Hu mice were indistinguishable from WT control mice in appearance, body weight, lifespan, and fertility (data not shown). Further assessment of T cells, B cells, dendritic cells (DCs), macrophages and granulocytes in the thymus, spleen, and peripheral lymph nodes (LNs) revealed no significant difference (FIG. 2A, 2B, 2C in FIG. 2), consistent with observations from a previous study. We then injected luciferase-expressing GL261 cells into mice intracranially to model human glioblastoma. In comparison with the WT group, IL8-Hu mice transplanted with GL261 displayed significantly larger tumor sizes and shorter lifespans (FIG. 3A and 3B in FIG. 3). Histological examination of tumor tissue from IL8-Hu mice by IHC and H&E staining showed an increase in tumor vascular vessel densities (FIG. 3D in FIG. 3). More importantly, flow cytometry analysis of tumors from IL8-Hu mice showed that IL-8 expression was greatly increased in CD4⁺ T cells and more profoundly increased in the CD45⁺ CD3⁻ myeloid cell compartment, confirming the results obtained from human samples (FIG. 3C in FIG. 3). Notably, tumor-infiltrating CD4⁺ and CD8⁺ T cells from IL8-Hu mice carried more of the exhaustion markers PD-1 and TIM-3 than those from the control group (FIG. 4A and 4B in FIG. 4). Therefore, in comparison with WT mice, our newly generated IL8-Hu mouse strain is more suitable to model human glioma, in which physiological expression of human IL-8 enables the recapitulation of tumor growth acceleration and angiogenesis.

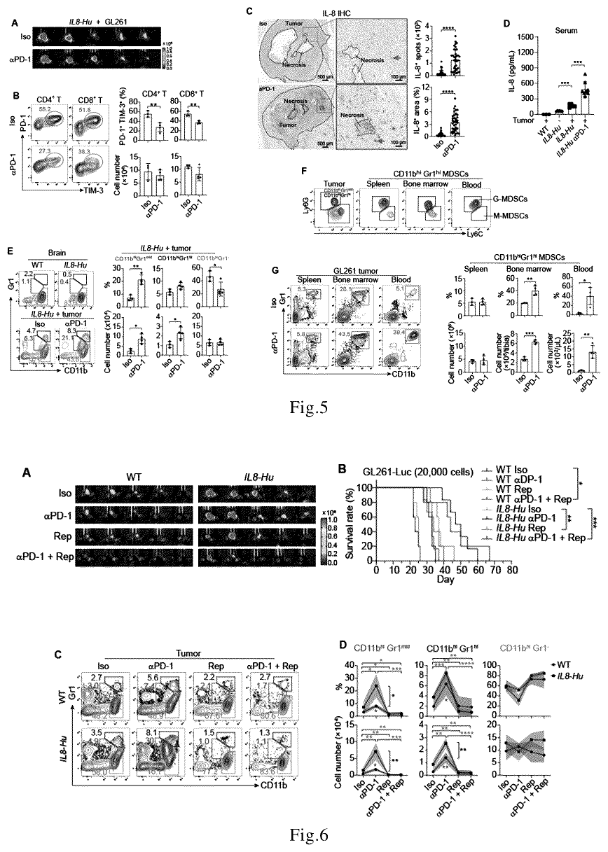

There is growing awareness that IL-8-enforced immunosuppression remains a hurdle for favorable outcomes of ICB treatment in several tumor types. By taking advantage of our newly developed IL8-Hu mouse model, we sought to explore whether IL-8 is a potential factor accountable for the low efficacy of anti-PD-1 in malignant glioma. We conducted a tumor implantation assay similar to that shown in FIG. 5C by intracranially injecting luciferase-expressing GL261 cells into IL8-Hu mice. Upon tumor establishment, tumor-bearing mice were subjected to intraperitoneal (i.p.) administration of anti-PD-1antibody or isotype IgG. In agreement with previous observations from monotherapy, anti-PD-1 treatment did not show a favorable antitumor response, even though T-cell exhaustion in the tumors was significantly alleviated by anti-PD-1 treatment (FIG. 5A and 5B in FIG. 5). Unexpectedly, the assessment of IL-8 levels in these mice showed that anti-PD-1 treatment significantly enhanced IL-8 expression in both sera and tumor sites compared to the control group (FIG. 5C and 5D in FIG. 5). Given that human IL-8 is able to chemoattract murine CD11b⁺ myeloid cells, we analyzed CD11b⁺ myeloid cell populations from the tumor sites and found significant enhancement of two populations of CD45⁺ CD11b^{hi} Gr1^{mid} and CD45⁺CD11b^{hi} Gr1^{hi} MDSCs in anti-PD-1-treated mice compared to the control group, while CD11b^{hi} Gr1⁻ cells, mainly composed of monocyte/microglia and dendritic cells, showed no significant change in cellularity (FIG. 5E in FIG. 5). Phenotypic characterization of the two populations of MDSCs at the tumor sites defined CD11b^{hi} Gr1^{mid} MDSCs as monocytic (M)-MDSCs signatured with Ly6C⁺, while CD11b^{hi} Gr1^{hi} cells contained both Ly6G⁺ G-MDSCs and Ly6C⁺ M-MDSCs, consistent with previous findings (FIG. 5F in FIG. 5). In contrast to the tumor sites, only CD11b^{hi} Gr1^{hi} MDSCs, composed mainly of Ly6G⁺ G-MDSCs, were observed in the spleen, bone marrow, and blood, and they were significantly increased in the bone marrow and blood but not in the spleen from anti-PD-1-treated mice in comparison with control mice (FIG. 5F and 5G in FIG. 5). Because the MDSC population was profoundly augmented in both peripheral blood and tumor sites, when assessing IL-8 production in MDSCs, we found that those from the spleen, blood and tumor sites, but not from the bone marrow, produced large amounts of IL-8, suggesting that MDSCs and IL-8 might play reciprocal roles in facilitating systemic immunosuppression in tumor-bearing mice and cancer patients. Collectively, these findings demonstrated that anti-PD-1 treatment in murine glioma could increase IL-8 expression and MDSC recruitment systemically, which in turn blunted anti-PD-1 treatment efficacy.

Whether the blockade of the IL-8-CXCR1/CXCR2 axis could potentiate anti-PD-1 efficacy in malignant glioma was investigated. We utilized reparixin, a described pharmacological inhibitor of CXCR1 and CXCR2, to test this idea. As previously described, luciferase-expressing GL261 cells were intracranially implanted into WT and IL8-Hu mice for seven days, followed by i.p. administration of isotype IgG, anti-PD-1 antibody, reparixin, and reparixin plus anti-PD-1. Again, anti-PD-1 monotherapy did not inhibit tumor growth in either IL8-Hu or WT mice (FIG. 6A and 6B in FIG. 6). Treatment with reparixin resulted in significantly slower tumor growth and improved survival in both IL8-Hu and WT mice almost equally (FIG. 6A and 6B in FIG. 6). More strikingly, both IL8-Hu and WT mice that received double administration of anti-PD-1 with reparixin showed the best outcome with respect to reduced tumor burden and extended survival (FIG. 6A and 6B in FIG. 6). Flow cytometry assessment revealed that reparixin treatment abolished not only anti-PD-1-induced but also basal levels of both CD11b^{hi} Gr1^{hi} MDSCs and CD11b^{hi} Gr1^{mid} MDSCs in tumors and to a lesser extent from WT mice (FIG. 6C, 6D in FIG. 6). Therefore, our results provide evidence to support the idea that the sequestration of MDSCs by targeting CXCR1 and CXCR2 could leverage anti-PD-1 therapeutic efficacy for glioma.

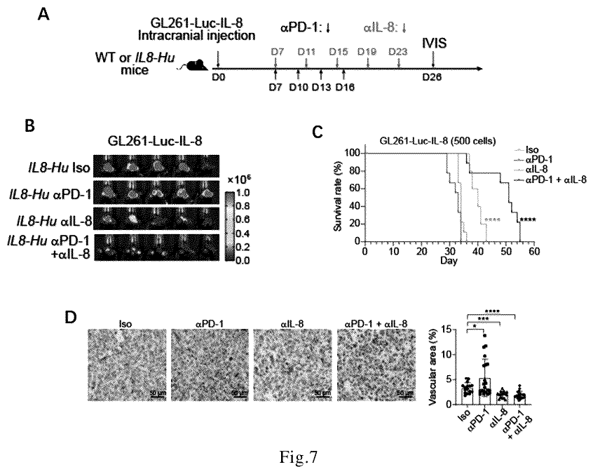

our study was performed by examining whether the neutralization of IL-8 has a similar effect. Because human primary glioblastoma cells also express IL-8 at high levels, we stringently modeled human glioblastoma by injecting GL261 cells with dual expression of IL-8 and luciferase into IL8-Hu mice, which were then subjected to sequential treatment with control isotype antibody, single blockade of PD-1 or IL-8, or dual blockade of PD-1 plus IL-8, as shown in FIG. 6F. Compared to mice that received isotype antibody, mice that received anti-IL-8 antibody showed significantly smaller tumor size and improved survival (FIG. 7A, 7B and 7C in FIG. 7). Notably, dual blockade of PD-1 and IL-8 substantially extended the median survival by ˜18 days compared to that of the isotype control group (FIG. 7C in FIG. 7). Given our aforementioned data showing that anti-PD-1 antibody administration augmented systemic IL-8 in IL8-Hu mice and the fact that IL-8 is capable of promoting angiogenesis in tumors, we conducted histological assessment of microvascular vessels in glioma tumors by IHC staining of CD31. Compared to control mice, tumor-bearing IL8-Hu mice that received anti-PD-1 antibody showed significantly higher microvascular densities, which were then greatly reduced by anti-IL-8 treatment (FIG. 7D in FIG. 7). Altogether, our findings suggested that the neutralization of IL-8, equivalent to the blockade of its receptors CXCR1 and CXCR2, functions to enhance T-cell-mediated antitumor immunity by abolishing suppressive MDSCs.

All publications mentioned herein are incorporated by reference as if each individual document was cited as a reference, as in the present application. It should also be understood that, after reading the above teachings of the present invention, those skilled in the art can make various changes or modifications, equivalents of which falls in the scope of claims as defined in the appended claims.

1.-10. (canceled)

11. An antibody, wherein the antibody comprises:
(1) the heavy chain variable region; and/or
(2) the light chain variable region;
the heavy chain variable region comprises the following three complementary determining regions (CDRs):
CDR1 as shown in SEQ ID NO: 1,
CDR2 as shown in SEQ ID NO: 2, and
CDR3 as shown in SEQ ID NO: 3;
the light chain variable region comprises the following three complementary determining regions (CDRs):
CDR1′ as shown in SEQ ID NO:4,
CDR2′ as shown in SEQ ID NO: 5, and
CDR3′ as shown in SEQ ID NO:6.

12. The antibody of claim 11, wherein the heavy chain variable region has an amino acid sequence as shown in SEQ ID NO: 7; and/or
the light chain variable region has an amino acid sequence as shown in SEQ ID NO: 8.

13. The antibody of claim 11, wherein the antibody is anti-IL-8 antibody.

14. The antibody of claim 13, wherein the IL-8 is human IL-8.

15. The antibody of claim 11, wherein any one of the amino acid sequences further comprises a derived sequence in which at least one amino acid (for example, 1-3, preferably 1-2, more preferably 1) is optionally added, deleted, modified and/or substituted.

16. The antibody of claim 15 wherein the derived sequence can retain the binding affinity of IL-8.

17. The antibody of claim 11, the heavy chain of the antibody further comprises a heavy chain constant region; and
the light chain of the antibody further comprises a light chain constant region.

18. The antibody of claim 17, wherein the heavy chain constant region is humanized, murine or rabbit origin; and
the light chain constant region is human, murine or rabbit origin.

19. The antibody of claim 11, wherein the antibody is selected from the group consisting of animal-derived antibodies, chimeric antibodies, humanized antibodies, and combinations thereof.

20. A polynucleotide, wherein the polynucleotide encodes the antibody of claim 11.

21. A method for blocking or inhibiting IL-8 induced activities; and/or preventing and/or treating the diseases associated with the IL-8, which comprises administering the antibody of claim 11 to a subject in need, thereby blocking or inhibiting IL-8 induced activities; and/or preventing and/or treating the diseases associated with the IL-8.

22. The method of claim 21, the IL-8 induced activities comprises proinflammatory activities, chemotactic activities, and/or angiogenesis;
the diseases associated with the IL-8 comprises autoimmune, inflammatory, infectious diseases and/or tumor;
the diseases associated with the IL-8 is the diseases associated with the elevated or unbalanced level of IL-8;
the diseases associated with the IL-8 is immune, autoimmune, inflammatory, infectious diseases or disorders characterised by elevated or unbalanced level of human IL-8, particularly rheumatoid arthritis, ulcerative colitis, asthma, chronic obstructive pulmonary disease (COPD), gout, cancer, flu, acne, inflammatory bowel disease (IBD), psoriasis, sepsis, osteoarthritis, erosive arthritis, atherosclerosis, transplant rejection, acute lung disease, acute lung injury, acute respiratory distress syndrome (ARDS), Crohn's disease, peripheral artery disease, systemic sclerosis, deep vein thrombosis, meningitis, encephalitis, uveitis, endometriosis, cystic fibrosis, diffuse panbronchiolitis, reperfusion injury, cystic fibrosis, vasculitis, familial Mediterranean fever, nephritis, chronic renal failure, juvenile onset diabetes, purpura, acute pancreatitis;
the diseases associated with the IL-8 is inflammatory or skin disorders, such as psoriasis, pustulosis palmoplantaris (PPP), bullous pemphigoid, pemphigus, contact dermatitis, eczema, erythematosus, and atopic dermatitis; and/or
the diseases associated with the IL-8 is human virus associated diseases or infections, such as common cold as caused by human rhinovirus, coronavirus, other enterovirus, herpes virus, influenza virus, parainfluenza virus, respiratory syncytial virus or adenovirus infection and hepatitis C.
